(19) United States
(12) Patent Application Publication
(10) Pub. No.: US 2012/0252865 A1
(43) Pub. Date: Oct. 4, 2012

(54) CARBAMATE DERIVATIVES IN PARTICULAR FOR THE TREATMENT OF NEUROLOGICAL DISORDERS

(75) Inventors: Walter Cabri, Rozzano (MI) (IT); Patrizia Minetti, Rome (IT); Giuseppe Campiani, Chianciano Terme (SI) (IT); Stefania Butini, Siena (IT)

(73) Assignee: SIGMA-TAU INDUSTRIE FARMACEUTICHE RIUNITE S.P.A., ROME (IT)

(21) Appl. No.: 13/202,133

(22) PCT Filed: Mar. 8, 2010
(86) PCT No.: PCT/EP10/52884

§ 371 Date: Oct. 17, 2011

(30) Foreign Application Priority Data
Mar. 18, 2009 (EP) 09155528.4

(51) Int. Cl. A61K 31/40; A61K 31/341; C07D 207/34; C07D 333/22; C07D 307/68; A61P 25/00; A61P 3/00; A61P 9/00; A61P 11/00; A61P 1/16; A61P 1/00; A61P 35/00; A61P 21/00; A61P 25/22; A61P 25/16; A61K 31/38

(52) U.S. Cl. 514/423; 514/448; 514/471; 548/531; 548/537; 549/72; 549/486; 549/487

(57) ABSTRACT

The present invention relates to new carbamate derivatives of formula I, processes for their preparation, and to pharmaceutical compositions containing them for the treatment of neurological disorders, such as neuropathic pain and anxiety.

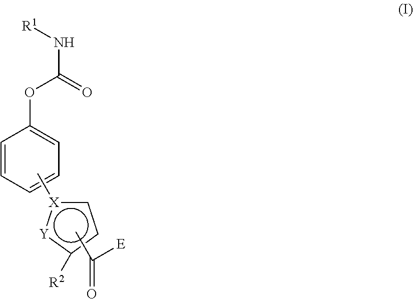

### FIELD OF THE INVENTION

The present invention relates to new carbamate derivatives, processes for their preparation, and to pharmaceutical compositions containing them for the treatment of neurological disorders, such as neuropathic pain and anxiety.

### BACKGROUND OF THE INVENTION

Anandamide and other fatty acid amides are known to be chemical messengers that modulate a number of physiological processes (Hanus L. O., Chem. Biodivers., 2007, 4, 1828). Anandamide activates through binding both the central-type (CB1) and peripheral type (CB2) cannabinoid receptors (Devane W. A., et al., Science, 1992, 258, 1946-1949). Anandamide has been reported to be implicated in the modulation of nociception, feeding, emesis, anxiety, cell proliferation, inflammation, and memory (Labar G., et al., Chem. Biodivers., 2007, 4, 1882).

The pharmacological action of anandamide is terminated by fatty-acid-amide-hydrolase (FAAH), an enzyme distributed in the central nervous system that degrades fatty acid amides at their site of action (Cravatt B. F., et al., Nature, 1996, 384, 83; Patricelli M. P., et al., Biochemistry, 1999, 38, 9804; WO 98/20119 and U.S. Pat. No. 6,271,015). The crystal structure of a complex of FAAH with a ligand has been solved, confirming that it exerts its catalytic action via the triad Ser-Ser-Lys (Bracey M. H., et al., Science, 2002, 298, 29, 1793).

FAAH is also responsible of the catabolism of many other lipid signaling fatty acid amides (i.e. oleamide, N-oleoylethanolamine, arachidonylglycerol and palmitoylethanolamide). Modulating the activity of the endocannabinoid system by restoring the levels of endogenous signaling lipids turned out to hold therapeutic promise in a wide range of disparate diseases and pathological conditions such as diseases of energy metabolism (cachexia and anorexia), pain and inflammation, central nervous system disorders (stroke, multiple sclerosis, Parkinson's disease, Huntington disease, Alzheimer disease, epilepsy, schizophrenia, anxiety, depression and insomnia), cardiovascular and respiratory disorders (hypertension, circulatory shock, myocardial reperfusion injury, atherosclerosis and asthma), retinopathy, cancer, gastrointestinal and liver disorders (inflammatory bowel disease and hepatitis), musculoskeletal disorders (arthritis and osteoporosis) as nicely reviewed lately (Pasher P., et al., Pharmacol. Rev., 2006, 58, 389 and references therein).

FAAH−/− KO mice cannot metabolize anandamide and, though fertile and generally normal, show signs of enhanced anandamide and related fatty acid amides activity at cannabinoid receptors, such as reduced pain sensation (Cravatt B. F., et al., Proc. Natl. Acad. Sci., 2001, 98, 9371). This suggests the possibility that drugs targeting FAAH may heighten the tonic action of anandamide, while possibly avoiding the multiple, often unwanted effects produced by Δ⁹-THC and other direct-acting cannabinoid agonists (Hall W., et al., Lancet, 1998, 352, 1611; Chaperon, F., et al., Crit. Rev. Neurobiol., 1999, 13, 243).

In particular URB-597, an irreversible carbamate-based inhibitor, was reported to be efficacious in the zero plus maze animal model of anxiety as well as to have analgesic efficacy in the rat hot plate and formalin tests (Kathuria S., et al, Nat. Med., 2003, 9, 1, 76). This compound is, among other derivatives, the object of the application WO04033422. Even though this application presents a general formula broadly claiming a multitude of structurally different compounds, it does not disclose nor suggest any of the compounds of the present invention. Indeed, support can mainly be found with regard to biphenyl derivatives. URB-597 was in fact identified through optimization of the lipophilic biphenyl derivative URB-524 by substituting the biphenyl scaffold that was recognized, via 3D-QSAR model, as being crucial for conferring activity (Tarzia G., et al., J. Med. Chem., 2003, 46, 12, 2352). WO08013963 describes fatty acid amide hydrolase inhibitors of general formula RXY wherein carbamate derivatives are encompassed. The most potent compounds however, appear to be the keto-oxadiazole derivatives; the most potent of which demonstrated a 15 nm activity with respect to a reported 4 μm activity for the most potent carbamate adduct. None of the compounds of the present invention are described nor suggested in the above application. The application WO03051842 relates to compositions decreasing activity of hormone-sensitive lipase containing compounds of formula 1.

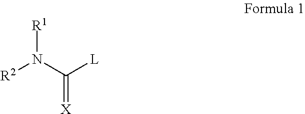

wherein R¹ can be H, substituted or not alkyl, alkenyl or cycloalkyl; X can be O or S; R² can have a wide variety of meanings comprising the ones corresponding to R¹, and L is a hydrolysable group. However, none of the compounds of the present invention are described nor suggested in the above application.

WO08129129 disclosed heterocyclic carbamates as novel FAAH inhibitors having the general formula 2 wherein the preferred compounds have a R substituent preferably comprised within the group consisting of the following radicals: methoxycarbonyl, oxazolyl, tetrazolyl, thiadiazolyl, benzoxazole-carbonyl and benzothiazolecarbonyl. However, besides the fact that the alleged in vivo activity and/or selectivity profile are not supported by any biological experiment, such compounds are not structurally related to the compounds of the present invention.

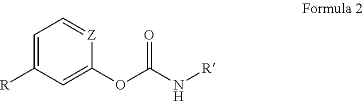

The potential therapeutic relevance of inhibiting FAAH has stimulated interest in developing selective and potent inhibitors. Such a strategy potentially represents a safer alternative to the use of exogenous cannabinoid agonists, which have been found to give variable effects. Inhibiting FAAH seems an ideal way of elevating the levels of the endogenous amidated lipids that activate CB1 receptors. Therefore, the desire of potent and selective FAAH inhibitors remains an interesting and promising goal.

### DESCRIPTION OF THE INVENTION

The invention provides novel compounds for inhibiting Fatty Acid Amide Hydrolase (FAAH), compositions that include such compounds as well as methods of treating diseases of energy metabolism, pain and inflammation, central nervous system disorders, cardiovascular and respiratory disorders, retinopathy, cancer, gastrointestinal and liver disorders and musculoskeletal disorders by administering FAAH inhibitors to a patient.

The invention comprises compounds of general formula I

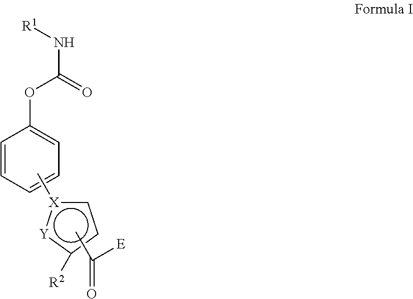

wherein,

R¹ is H, (C₁-C₄)-alkyl, (C₃-C₆)-cycloalkyl, (C₁-C₆)-alkyl substituted with aryl or (C₂-C₅)-alkynyl;

X is C or N;

Y is CH, O or S;

R² is H or (C₁-C₄)-alkyl;

E is NR³R⁴ or OR⁵;

R³ and R⁴, the same or different are H or (C₂-C₆)-alkyl optionally substituted with aryl;

R⁵ is (C₂-C₆)-alkyl optionally substituted with aryl or with (C₂-C₅)-alkynyl;

wherein the cycle containing the radicals X and Y is a heteroaromatic ring; its optically active forms such as enantiomers, diastereomers and its racemate forms, as well as pharmaceutically acceptable salts thereof;
with the proviso that when X is N, Y is CH.

An embodiment of this invention is that of compounds of formula I, for use as medicaments.

In a further embodiment, said medicament is used for treating a neurological disorder, diseases of energy metabolism, cardiovascular and respiratory disorders, gastrointestinal and liver disorders, retinopathy, cancer and musculoskeletal disorders.

In a preferred embodiment, said medicament is used for treating a neurological disorder.

In a more preferred embodiment, said medicament is used for treating anxiety and pain.

The term “alkyl” refers to linear or branched alkyl groups having preferably from 1 to about 12 carbon atoms. Lower alkyl group is exemplified by groups such as methyl, ethyl, n-propyl, isopropyl, n-butyl, isobutyl, tert-butyl, pentyl, iso-pentyl, neo-pentyl, n-hexyl and the like.

The term “alkoxy” refers to a group —OR where R includes lower alkyl, “C₃-C₁₀ cycloalkyl” and “heterocycloalkyl”.

The terms “heterocycloalkyl” and/or heterocycle refer to a saturated five- or six-membered ring containing one or two nitrogen, oxygen or sulfur atoms.

Preferred heterocycloalkyl include pyrrolidine, piperidine, piperazine, morpholine, thiomorpholine and the like.

The term “aryl” refers to an aromatic carbocyclic group of 6 to 14 carbon atoms having a single ring (e.g., phenyl) or multiple rings, which may be attached in a pendent manner or may be fused. Preferred aryl include phenyl, naphthyl, biphenyl, indane and the like.

The term “heteroaryl” refers to a monocyclic heteroaromatic, or a bicyclic fused-ring heteroaromatic group. Particular examples of heteroaromatic groups include optionally substituted pyridyl, pyrrolyl, furyl or thienyl.

The term “aminocarbonyl” refers to the group —C(O)NRR′ where each R, R′ includes independently H, “alkyl”, “aryl” or “arylaminocarbonyl”.

“Pharmaceutically acceptable salts” refers to salts of the below identified compounds of formulae (I), that retain the desired biological activity. Examples of such salts include, but are not restricted to acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, toluene sulfonic acid, naphthalene disulfonic acid, methanesulfonic acid and poly-galacturonic acid. When the salt is of a mono acid (for example, the hydrochloride, the hydrobromide, the p-toluenesulphonate, or the acetate), the hydrogen form of a di-acid (for example, the hydrogen sulphate, or the succinate), or the dihydrogen form of a tri-acid (for example, the dihydrogen phosphate, or the citrate), at least one molar equivalent and usually a molar excess of the acid is employed. However, when such salts as the sulphate, the hemisuccinate, the hydrogen phosphate, or the phosphate are desired, the appropriate and exact chemical equivalents of acid are generally used. Suitable pharmaceutically acceptable base addition salts for the compound of the present invention include metallic salts made from aluminium, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Sodium salts are particularly preferred.

An embodiment of this invention is that of compounds of formula (I) described earlier, wherein E is NR³R⁴ with R³ and R⁴ being H.

Another embodiment of this invention is that of compounds of formula (I) described earlier, wherein R¹ is (C₁-C₆)-alkyl substituted with aryl or (C₂-C₅)-alkynyl.

The compounds of the present invention can be prepared by conventional synthetic methods and are described underneath. It will be appreciated that where typical or preferred experimental conditions (i.e. reaction temperatures, time, moles of reagents, solvents, etc.) are given, other experimental conditions can also be used, unless otherwise stated.

The invention furthermore provides a process for the preparation of compounds of formula I, which can be obtained by reacting compounds of formula II,

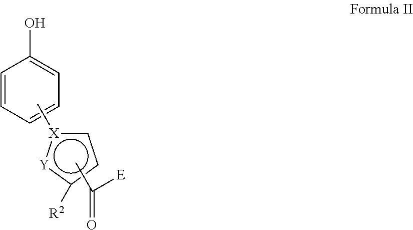

wherein X, Y, E and R² are as described above, with compounds of formula III

R¹—N═C═O Formula III

in a polar solvent in the presence of a base such as NEt₃.

In all said transformations, any interfering reactive group can be protected and then deprotected according to well-established procedures described in organic chemistry (see for example: Greene T. W., Wuts P. G. M., “Protective Groups in Organic Synthesis”, J. Wiley & Sons, Inc., 3^{rd} Ed., 1999) and well known to those skilled in the art.

All said transformations are only examples of well-established procedures described in organic chemistry (see for example: March J., “Advanced Organic Chemistry”, J. Wiley & Sons, Inc., 4^{th} Ed., 1992) and well known to those skilled in the art.

We have found that the derivatives (I) and their pharmaceutically acceptable salts, prepared according to the invention, are useful agents for the treatment of disease states, disorders and pathological conditions mediated by fatty acid amide hydrolase; in particular for the treatment of anxiety and pain.

Therefore another object of the present invention is a method of treating a mammal suffering from disease states, disorders and pathological conditions mediated by fatty acid amide hydrolase; in particular of anxiety and pain, comprising administering a therapeutically effective amount of a compound of Formula (I) as described above.

The term “therapeutically effective amount” as used herein refers to an amount of a therapeutic agent needed to treat, ameliorate a targeted disease or condition, or to exhibit a detectable therapeutic effect.

For any compound, the therapeutically effective dose can be estimated initially either in cell culture assays or in animal models, usually mice, rats, guinea pigs, rabbits, dogs, or pigs. The animal model may also be used to determine the appropriate concentration range and route of administration. Such information can then be used to determine useful doses and routes for administration in humans. In calculating the Human Equivalent Dose (HED) it is recommended to use the conversion table provided in Guidance for Industry and Reviewers document (2002, U.S. Food and Drug Administration, Rockville, Md., USA).

The pharmaceutical compositions will contain at least one compound of Formula (I) as an active ingredient, in an amount such as to produce a significant therapeutic effect. The compositions covered by the present invention are entirely conventional and are obtained with methods which are common practice in the pharmaceutical industry, such as, for example, those illustrated in Remington's Pharmaceutical Science Handbook, Mack Pub. N.Y.—last edition. According to the administration route chosen, the compositions will be in solid or liquid form, suitable for oral, parenteral or topical administration. The compositions according to the present invention contain, along with the active ingredient, at least one pharmaceutically acceptable vehicle or excipient. These may be particularly useful formulation coadjuvants, e.g. solubilising agents, dispersing agents, suspension agents, and emulsifying agents.

The amount of the compound actually administered will typically be determined by a physician, in the light of the relevant circumstances, including the condition to be treated, the chosen route of administration, the actual compound administered, drug combination, the age, body weight, and response of the individual patient, the severity of the patient's symptoms, and the like. Generally, an effective dose will be from 0.01 mg/kg to 100 mg/kg, preferably 0.05 mg/kg to 50 mg/kg. For any compound, the therapeutically effective dose can be estimated initially either in cell culture assays or in animal models, usually mice, rats, guinea pigs, rabbits, dogs, or pigs. The precise effective dose for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician.

Compositions may be administered individually to a patient or may be administered in combination with other agents, drugs or hormones.

The medicament may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity.

Suitable carriers may be large, slowly metabolised macromolecules such as proteins, polysaccharides, polylactic acids, polyglycolic acids, polymeric amino acids, amino acid copolymers and inactive virus particles.

A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol.

Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The medicament of this invention may be administered by any number of routes including, but not limited to, oral, intravenous, intramuscular, intra-arterial, intramedullary, intrathecal, intraventricular, transdermal or transcutaneous applications, subcutaneous, intraperitoneal, intranasal, enteral, topical, sublingual, intravaginal or rectal means.

The compositions for oral administration may take the form of bulk liquid solutions or suspensions, or bulk powders. More commonly, however, the compositions are presented in unit dosage forms to facilitate accurate dosing.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

Typical unit dosage forms include refilled, pre-measured ampoules or syringes of the liquid compositions or pills, tablets, capsules or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form. Dosage treatment may be a single dose schedule or a multiple dose schedule.

An object of the present invention are pharmaceutical compositions containing one or more of the compounds of formula (I) described earlier, in combination with excipients and/or pharmacologically acceptable diluents.

The compositions in question may, together with the compounds of formula (I), contain known active principles.

A further object of the invention is a process for the preparation of pharmaceutical compositions characterised by mixing one or more compounds of formula (I) with suitable excipients, stabilizers and/or pharmaceutically acceptable diluents.

### DESCRIPTION OF THE DRAWING

FIG. 1: it describes the analgesic effect of the selective FAAH inhibitors ST4068, ST3911 and ST3913 at a dose of 30 mg/kg, together with the reference compound URB597 at a dose of 50 mg/kg.

FIG. 2: it describes the dose response of the analgesic effect of the selective FAAH inhibitor ST4068 at the doses of 10, 30 and 100 mg/kg.

FIG. 3: it describes the dose response of the analgesic effect of the selective FAAH inhibitor ST3911 at the doses of 10, 30 and 100 mg/kg.

FIG. 4: it describes the dose response of the analgesic effect of the selective FAAH inhibitor ST3913 at the doses of 10, 30 and 100 mg/kg.

### ABBREVIATIONS

AA: arachidonic acid
AcOEt: ethyl acetate
AnNH: arachidonoylethanolamide (anandamide)
bd: broad doublet
bs: broad singlet
CH₂Cl₂: dichloromethane
DMSO: dimethylsyulfoxide
Et₂O: diethyl ether
NaH: sodium hydride
NaOH. Sodium hydroxide
NEt₃: triethylamine
NH₄OH: ammonium hydroxide
RP-HPLC: reversed phase-high-performance liquid chromatography
RT: room temperature
SOCl₂: thionyl chloride
THF: tetrahydrofuran

General Remarks: 1H spectra were recorded in CDCl₃ solution as indicated, at 300 MHz with a Bruker instrument. The chemical shift values are given in ppm and the coupling constants in Hz. Flash column chromatography was carried out using silica gel (Merck 230-400 mesh).

### Example 1

### butyl-carbamic acid 3-(3-carbamoyl-pyrrol-1-yl)-phenyl ester ST3910

### STEP 1: 1-(3-hydroxy-phenyl)-1H-pyrrole-3-carbaldehyde

To a solution of m-aminophenol (588.0 mg, 5.39 mmol) in acetic acid (10.0 ml) and water (2.0 ml), dimethoxytetrahydrofuran carboxaldehyde (950.0 mg, 5.93 mmol) in acetic acid (1.0 ml) was added dropwise. The solution was heated to 100° C. for 15 min. After removal of the solvent, the dark brown reaction mixture was diluted with AcOEt and neutralized with an aqueous saturated sodium carbonate solution. The latter was extracted with ethyl acetate (3×100 ml), dried over sodium sulfate, filtered and concentrated. Column chromatography (1:1 n-hexane/AcOEt) afforded the pure product as a yellow solid (42% yield).

¹H NMR (acetone-d₆) δ: 6.70 (s, 1H), 6.87 (d, 1H, J=7.8 Hz), 7.08-7.12 (m, 2H), 7.35-7.37 (m, 2H), 8.00 (s, 1H), 8.84 (s, 1H), 9.84 (s, 1H).

ESI-MS m/z [M−H]⁺ 186.

### STEP 2: 1-(3-hydroxy-phenyl)-1H-pyrrole-3-carboxylic acid

Silver nitrate (370.0 mg, 2.18 mmol) was added to a solution of 1-(3-hydroxy-phenyl)-1H-pyrrole-3-carbaldehyde (255.0 mg, 1.36 mmol) in 3.0 ml of methanol and 3.0 ml of 6N NaOH. The reaction mixture was then stirred at reflux for 1 h. After cooling, the solvent was removed. The residue was extracted with AcOEt. The aqueous layer was acidified to pH 1 using concentrated HCl. The latter was then extracted with AcOEt (3×20 ml). The organic layer was then dried over sodium sulfate, filtered and concentrated to afford the product as a pure white solid (73% yield).

¹H NMR (acetone-d₆) δ: 6.67 (dd, 1H, J=1.8, 3.0 Hz), 6.84 (ddd, 1H, J=7.8, 2.1, 0.9 Hz), 7.04-7.11 (m, 2H), 7.25 (dd, 1H, J=2.1, 3.0 Hz), 7.34 (t, 1H, J=8.4 Hz), 7.78 (dd, 1H, J=1.8, 2.4 Hz,), 8.78 (s, 1H), 10.49 (brs, 1H).

ESI-MS m/z [M−H]⁺ 202

### STEP 3: 1-(3-hydroxy-phenyl)-1H-pyrrole-3-carboxylic acid amide

1-(3-hydroxy-phenyl)-1H-pyrrole-3-carboxylic acid (180.0 mg, 0.87 mmol) was added portion wise to 1.5 ml of SOCl₂ at RT. The reaction mixture was stirred at reflux for 30 min. After cooling, it was evaporated to dryness. The resulting residue was dissolved in 3.0 ml of THF and 5.0 ml of conc. NH₄OH were added cautiously at RT. The mixture was stirred for 7 h, then extracted with AcOEt, dried over sodium sulfate, filtered and concentrated. Column chromatography (95:5 CHCl₃/MeOH) afforded 100.0 mg of the pure product as a brown viscous oil (57% yield).

¹H NMR (acetone-d₆) δ: 6.47 (brs, 1H), 6.66 (brs, 1H), 6.76-6.84 (m, 2H), 6.99-7.04 (m, 2H), 7.20-7.31 (m, 2H), 7.85 (s, 1H), 9.40 (brs, 1H).

### STEP 4: butyl-carbamic acid 3-(3-carbamoyl-pyrrol-1-yl)-phenyl ester ST3910

n-butylisocyanate (90 μl, 0.79 mmol) and NEt₃ (110 μl, 0.79 mmol) were added to a solution of 1-(3-hydroxy-phenyl)-1H-pyrrole-3-carboxylic acid amide (40.0 mg, 0.20 mmol) in 2.0 ml of dry THF. The reaction mixture was stirred at RT for 16 h. The solvent was removed and the crude reaction mixture was purified by flash chromatography (98/2 CHCl₃/MeOH) to lead 38 mg of the desired adduct (63% yield).

¹H NMR (acetone-d₆) δ: 0.94 (t, 3H, J=7.2 Hz), 1.34-1.47 (m, 2H), 1.52-1.62 (m, 2H), 3.22 (q, 2H, J=6.6 Hz), 6.35 (brs, 1H), 6.74 (s, 1H), 6.91 (brs, 2H), 7.08 (d, 1H, J=7.8 Hz), 7.28-7.50 (m, 4H), 7.85 (s, 1H).

¹³C NMR (acetone-d₆) δ: 13.4, 19.9, 32.0, 40.9, 110.6, 114.0, 116.6, 119.7, 120.2, 121.8, 122.7, 130.5, 140.9, 152.9, 154.3, 165.6.

Examples 2 to 4 were obtained following the experimental conditions described in example 1—step 4, using the adequate isocyanate reagents.

### Example 2

### undec-10-ynyl-carbamic acid 3-(3-carbamoyl-pyrrol-1-yl)-phenyl ester ST3911

Yield: 78%

¹H NMR (CD₃OD) δ: 1.35-1.56 (m, 14H), 2.13 (s, 1H), 2.16 (s, 2H), 3.18 (t, 2H, J=6.9 Hz), 6.72 (s, 1H), 7.08 (d, 1H, J=7.2 Hz), 7.22-7.39 (m, 3H), 7.48 (t, 1H, J=8.1 Hz), 7.80 (s, 1H).

¹³C NMR (CD₃OD) δ: 17.8, 26.7, 28.5, 28.6, 29.0, 29.2, 29.37, 29.5, 40.9, 68.2, 83.9, 110.2, 114.1, 117.0, 119.8, 120.5, 121.0, 122.3, 130.4, 140.9, 152.6, 155.5, 168.5.

### Example 3

### cyclohexyl-carbamic acid 3-(3-carbamoyl-pyrrol-1-yl)-phenyl ester ST3912

Yield: 62%

¹H NMR (CD₃OD) δ: 1.19-1.39 (m, 5H), 1.65 (d, 1H, J=12.6 Hz), 1.78 (d, 2H, J=11.7 Hz), 1.95 (d, 2H, J=10.8 Hz), 3.39-3.48 (m, 1H), 6.72 (s, 1H), 7.07 (d, 1H, J=7.8 Hz), 7.21-7.38 (m, 3H), 7.47 (t, 1H, J=7.8 Hz), 7.80 (s, 1H)

¹³C NMR (CD₃OD) δ: 25.0 (2), 25.4, 32.8 (2), 50.5, 110.2, 114.2, 116.9, 119.8, 120.5, 121.0, 122.3, 130.4, 140.8, 152.5, 154.7, 168.5.

### Example 4

### (6-phenyl-hexyl)-carbamic acid 3-(3-carbamoyl-pyrrol-1-yl)-phenyl ester ST3913

Yield: 60%

¹H NMR (CD₃OD) δ: 1.38-1.40 (m, 4H), 1.53-1.66 (m, 4H), 2.60 (t, 2H, J=15.0 Hz), 3.17 (t, 2H, J=13.8 Hz), 6.72 (s, 1H), 7.05-7.37 (m, 9H), 7.46 (t, 1H, J=8.4 Hz), 7.79 (s, 1H).

¹³C NMR (CD₃OD) δ: 26.5, 28.8, 29.5, 31.5, 35.6, 40.9, 110.2, 114.1, 117.0, 119.8, 120.5, 121.0, 122.3, 125.5, 128.1 (2), 128.2 (2), 130.4, 140.8, 142.7, 152.5, 155.5, 168.5.

ESI-MS m/z [M+H]⁺ 406, [M+Na]⁺ 428, [M+K]⁺ 444, [2 M+H]⁺ 811, [2 M+Na]⁺ 833.

### Example 5

### butyl-carbamic acid 3-(3-carbamoyl-5-methyl-furan-2-yl)-phenyl ester ST4067

### STEP 1: 2-(3-methoxy-benzoyl)-4-oxo-pentanoic acid ethyl ester

Ethyl 3-(3-methoxyphenyl)-3-oxopropanoate (262 ml, 1.35 mmol) was added dropwise to a suspension of NaH (36.0 mg, 1.49 mmol) in 8.0 ml of dry THF at 0° C. The reaction mixtured was stirred at this temperature for 30 min. Chloroacetone (119 ml, 1.49 mmol) was then added dropwise at 0° C. and the reaction mixture was stirred at RT for 48 h. The reaction was quenched with 1 N HCl (3.0 ml). THF was evaporated and the residue was extracted with AcOEt (3×25 ml). The combined organic layers were dried over sodium sulfate, filtered and concentrated. Column chromatography (3:1 n-hexane/AcOEt) afforded the pure product as a racemic mixture (67% yield).

¹H NMR (CDCl₃) δ: 1.14 (t, 3H, J=6.9 Hz), 2.21 (s, 3H), 3.06 (m, 2H), 3.83 (s, 3H), 4.10 (q, 2H, J=6.9 Hz), 4.85 (t, 1H, J=7.0 Hz), 7.09-7.13 (m, 1H), 7.33-7.39 (m, 1H), 7.51 (s, 1H), 7.59 (d, 1H, J=7.8 Hz).

### STEP 2: 2-(3-methoxy-phenyl)-5-methyl-furan-3-carboxylic acid ethyl ester

2-(3-methoxy-benzoyl)-4-oxo-pentanoic acid ethyl ester (735 mg, 2.64 mmol) was dissolved in 8.0 ml of pure EtOH. Concentrated HCl (0.73 ml) was added and the reaction mixture was carried on under MW irradiation (150 W) (CEM Discovery Microwave System) and refluxed (maximum internal temperature 100° C.) for 10 minutes. The reaction mixture was cooled and diluted with AcOEt, and was neutralized with saturated aqueous sodium bicarbonate and extracted with AcOEt (3×50 ml). The combined organic layers were dried over sodium sulfate, filtered and concentrated. Column chromatography (97:3 n-hexane/AcOEt) afforded the pure product as a colorless viscous oil (90% yield).

¹H NMR (CDCl₃) δ: 1.32 (t, 3H, J=6.9 Hz), 2.34 (d, 3H, J=0.9 Hz), 3.84 (s, 3H), 4.28 (q, 2H, J=7.2 Hz), 6.44 (d, 1H, J=1.2 Hz), 6.92 (ddd, 1H, J=0.6, 2.1, 8.1 Hz), 7.32 (t, 1H, J=7.8 Hz), 7.56-7.59 (m, 1H), 7.62-7.64 (m, 1H).

¹³C NMR (CDCl₃) δ: 13.5, 14.5, 55.5, 60.6, 109.2, 113.5, 115.0, 115.2, 120.7, 129.2, 131.4, 151.21, 155.8, 159.5, 163.9.

ESI-MS m/z [M−OEt]⁺ 215, [M+Na]⁺ 283.

### STEP 3: 2-(3-methoxy-phenyl)-5-methyl-furan-3-carboxylic acid

NaOH (161.0 mg, 4.03 mmol) was added to a solution of 2-(3-methoxy-phenyl)-5-methyl-furan-3-carboxylic acid ethyl ester (42.0 mg, 0.16 mmol) in EtOH (3.0 ml) and water (1.0 ml), and the reaction mixture was stirred at RT for 7 h. EtOH was then removed and the residue was acidified with 6N HCl to pH 1. The mixture was extracted with AcOEt (3×20 ml). The organic layers were dried over sodium sulfate, filtered and concentrated. The compound obtained (37.0 mg, 100% yield). was used in the next step without any further purification

¹H NMR (CDCl₃) δ: 2.37 (s, 3H), 3.86 (s, 3H), 6.48 (s, 1H), 6.95 (dd, 1H, J=7.8, 3.9 Hz,), 7.34 (t, 1H, J=7.9 Hz), 7.54-7.59 (m, 2H).

ESI-MS m/z [M−H]⁻ 231.

### STEP 4: 2-(3-methoxy-phenyl)-5-methyl-furan-3-carboxylic acid amide

2-(3-methoxy-phenyl)-5-methyl-furan-3-carboxylic acid (68.0 mg, 0.29 mmol) was added portion wise to 300 μl of SOCl₂ at RT. The reaction mixture was stirred at reflux for 30 min and, after cooling, was evaporated to dryness. The residue obtained was dissolved in 3.0 ml of THF and 1.0 ml of conc. NH₄OH were added cautiously at RT. The mixture was stirred for 7 h, then extracted with AcOEt, dried over sodium sulfate, filtered and concentrated. The crude reaction mixture was purified through column chromatography (95:5 CHCl₃/MeOH) to afford the pure product as a brown viscous oil (65.0 mg, 97% yield).

¹H NMR (CDCl₃) δ: 2.34 (d, 3H, J=0.6 Hz), 3.83 (s, 3H), 5.79 (brs, 1H), 5.90 (brs, 1H), 6.33 (d, 1H, J=0.9 Hz), 6.92 (dt, 1H, J=3.0, 7.5 Hz), 7.26-7.38 (m, 3H).

### STEP 5: 2-(3-hydroxy-phenyl)-5-methyl-furan-3-carboxylic acid amide

BBr₃ (1M solution in dichloromethane, 2.4 ml, 2.44 mmol) was added at −78° C. to a suspension of 2-(3-methoxy-phenyl)-5-methyl-furan-3-carboxylic acid amide (188.0 mg, 0.81 mmol) in dry CH₂Cl₂ (6.0 ml). The reaction mixture was then allowed to warm to RT and stirred for 8 h. A saturated aqueous solution of sodium carbonate was added to quench the reaction. The residue was extracted with AcOEt (3×20 ml). The combined organic extracts were dried over sodium sulfate, filtered and concentrated to afford the pure product as a brownish solid (175.0 mg, 98% yield).

¹H NMR (acetone-d₆) δ: 2.33 (d, 3H, J=0.9 Hz), 6.42 (d, 1H, J=0.9 Hz), 6.56 (brs, 1H), 6.81 (ddd, 1H, J=0.9, 2.7, 4.8 Hz), 6.94 (brs, 1H), 7.22 (t, 1H, J=7.8 Hz), 7.45 (dt, 1H, J=7.8, 1.5 Hz), 7.53 (t, 1H, J=1.8 Hz), 8.48 (s, 1H).

### STEP 6: butyl-carbamic acid 3-(3-carbamoyl-5-methyl-furan-2-yl)-phenyl ester

n-butylisocyanate (80 μl, 0.75 mmol) and NEt₃ (100 μl, 0.75 mmol) were added to a solution of 2-(3-hydroxy-phenyl)-5-methyl-furan-3-carboxylic acid amide (36.0 mg, 0.17 mmol), in 3.0 ml of dry THF. The reaction mixture was stirred at RT for 16 h. The solvent was removed and purification by means of column chromatography (99:1 CHCl₃/MeOH) afforded the pure product as a colorless oil (40.0 mg, 77% yield).

¹H NMR (CDCl₃) δ: 0.96 (t, 3H, J=7.5 Hz), 1.36-1.43 (m, 2H), 1.50-1.57 (m, 2H), 2.35 (d, 3H, J=0.9 Hz), 3.15-3.21 (m, 2H), 6.38 (d, 1H, J=0.9 Hz), 7.07 (ddd, 1H, J=0.9, 2.4, 4.8 Hz), 7.38 (t, 1H, J=7.8 Hz), 7.63 (t, 1H, J=1.8 Hz), 7.70 (dt, 1H, J=1.5, 5.4 Hz).

¹³C NMR (CDCl₃) δ: 12.0, 12.9, 19.8, 31.7, 40.5, 107.9, 118.2, 120.2, 121.6, 123.7, 129.0, 131.6, 151.4, 151.5, 151.9, 155.9, 168.0.

Example 6 was synthesized following the experimental conditions described in example 5—step 6, using phenylhexylisocyanate instead of n-butylisocyanate.

### Example 6

### (6-phenyl-hexyl)-carbamic acid 3-(3-carbamoyl-5-methyl-furan-2-yl)-phenyl ester ST4068

Yield: 71%

¹H NMR (CDCl₃) δ: 1.38 (brs, 4H), 1.55-1.66 (m, 4H), 2.33 (s, 3H), 2.61 (t, 2H, J=7.5 Hz), 3.24 (q, 2H, J=6.6 Hz), 5.10 (brs, 1H), 5.61 (brs, 1H), 5.82 (brs, 1H), 6.32 (s, 1H), 7.11-7.19 (m, 4H), 7.25-7.30 (m, 2H), 7.39 (d, 1H, J=7.8 Hz), 7.58 (s, 1H), 7.64 (d, 1H, J=7.8 Hz).

¹³C NMR (CDCl₃) δ: 13.6, 26.8, 29.1, 29.9, 31.6, 36.1, 41.5, 108.5, 118.1, 121.2, 122.3, 124.7, 125.9, 128.5 (2), 128.6 (2), 129.8, 131.3, 142.8, 151.3, 151.5, 152.1, 154.6, 165.9.

### Example 7

### (6-phenyl-hexyl)-carbamic acid 3-(3-carbamoyl-5-methyl-thiophen-2-yl)-phenyl ester ST4069

### STEP 1: 2-(3-methoxy-phenyl)-5-methyl-thiophene-3-carboxylic acid ethyl ester

Lawesson's reagent (2.00 g) was added to a solution of 2-(3-methoxy-benzoyl)-4-oxo-pentanoic acid ethyl ester (500.0 mg, 1.8 mmol) in 5.0 ml of toluene. The reaction was carried on under microwaves irradiation (150 W) (CEM Discovery Microwave System) under reflux (maximum internal temperature 100° C.) for minutes. After cooling, the mixture was filtered through Celite, and the solvent was removed under reduced pressure. Flash column chromatography (99:1 n-hexane/ethyl acetate) afforded the pure product as colorless oil (240.0 mg, 48% yield).

¹H NMR (CDCl₃) δ: 1.18 (t, 3H, J=7.2 Hz), 2.47 (d, 3H, J=0.9 Hz), 3.82 (s, 3H), 4.17 (q, 2H, J=6.9 Hz), 6.90 (ddd, 1H, J=0.9, 2.7, 8.4 Hz), 7.00-7.05 (m, 2H), 7.15 (d, 1H, J=0.9 Hz), 7.28 (t, 1H, J=7.8 Hz)

ESI-MS m/z [M−OEt]⁺ 231, [M+Na]⁺ 299.

### STEP 2: 2-(3-methoxy-phenyl)-5-methyl-thiophene-3-carboxylic acid

The compound was obtained following the experimental conditions described in example 5-step 3.

Yield: 97%. The compound was used in the next step without any purification.

¹H NMR (CDCl₃) δ: 2.46 (d, 3H, J=0.6 Hz), 3.81 (s, 3H), 6.92 (dd, 1H, J=2.4, 8.4 Hz), 7.03-7.07 (m, 2H), 7.19 (d, 1H, J=0.9 Hz), 7.28 (t, 1H, J=8.1 Hz).

### STEP 3: 2-(3-methoxy-phenyl)-5-methyl-thiophene-3-carboxylic acid amide

The compound was obtained following the experimental conditions described in example 5-step 4.

Yield: 78%.

¹H NMR (acetone-d₆) δ: 2.46 (d, 3H, J=0.6 Hz), 3.82 (s, 3H), 6.50 (brs, 2H), 6.93 (dd, 1H, J=1.8, 8.4 Hz), 6.99 (d, 1H, J=1.2 Hz), 7.06-7.11 (m, 2H), 7.32 (t, 1H, J=8.1 Hz).

### STEP 4: 2-(3-hydroxy-phenyl)-5-methyl-thiophene-3-carboxylic acid amide

The compound was obtained following the experimental conditions described in example 5-step 5.

Yield: 95%.

¹H NMR (acetone-d₆) δ: 2.45 (d, 3H, J=0.9 Hz), 6.49 (brs, 2H), 6.84 (ddd, 1H, J=0.9, 2.4, 8.1 Hz), 6.96-7.00 (m, 3H), 7.22 (t, 1H, J=7.5 Hz), 8.55 (s, 1H).

### STEP 5: (6-phenyl-hexyl)-carbamic acid 3-(3-carbamoyl-5-methyl-thiophen-2-yl)-phenyl ester

The compound was obtained following the experimental conditions described in example 5-step 6 reacting 2-(3-hydroxy-phenyl)-5-methyl-thiophene-3-carboxylic acid amide with phenylhexyl isocyanate.

Yield: 80%.

¹H NMR (CDCl₃) δ: 1.36-1.40 (m, 4H), 1.54-1.69 (m, 4H), 2.45 (d, 3H, J=1.2 Hz), 2.61 (t, 2H, J=7.5 Hz), 3.24 (q, 2H, J=6.9 Hz), 5.13 (t, 1H, J=5.4 Hz), 5.53 (brs, 2H), 7.12-7.19 (m, 5H), 7.25-7.32 (m, 4H), 7.39 (t, 1H, J=7.8 Hz).

¹³C NMR (CDCl₃) δ: 15.3, 26.8, 29.1, 29.9, 31.6, 36.1, 41.5, 122.3, 123.1, 125.9, 126.6, 127.9, 128.5 (2), 128.6 (2), 129.9, 132.6, 134.3, 139.5, 141.7, 142.8, 151.4, 154.5, 166.0.

### Example 8

### cyclohexyl-carbamic acid 3-(4-carbamoyl-5-methyl-furan-2-yl)-phenyl ester ST4104

### STEP 1: 2-[2-(3-methoxy-phenyl)-2-oxo-ethyl]-3-oxo-butyric acid ethyl ester

The compound was obtained following the experimental conditions described in example 5-step 1 using ethylacetoacetate and 2-bromo-1-(3-methoxy phenyl)ethanone instead of ethyl 3-(3-methoxyphenyl)-3-oxopropanoate and chloroacetone.

Yield: 79%.

¹H NMR (CDCl₃) δ: 1.29 (t, 3H, J=7.2 Hz), 2.45 (s, 3H), 3.51 (dd, 1H, J=18.6, 5.7 Hz), 3.71 (dd, 1H, J=18.6, 8.3 Hz), 3.85 (s, 3H), 4.23 (m, 3H), 7.12 (m, 1H), 7.37 (m, 1H), 7.47 (m, 1H), 7.58 (m, 1H); ESI-MS m/z [M+H]⁺ 279, [M+Na]+301 (100).

### STEP 2: 5-(3-methoxy-phenyl)-2-methyl-furan-3-carboxylic acid ethyl ester

The compound was obtained following the experimental conditions described in example 5-step 2.

Yield: 72%.

¹H NMR (CDCl₃) δ: 1.35 (t, 3H, J=7.2 Hz), 2.62 (s, 3H), 3.81 (s, 3H), 4.29 (q, 2H, J=7.2 Hz), 6.80 (m, 1H), 6.86 (s, 1H), 7.15-7.28 (m, 3H)

¹³C NMR (CDCl₃) δ: 14.1, 14.6, 55.4, 60.4, 106.0, 109.1, 113.6, 115.6, 116.4, 129.9, 131.5, 151.74, 158.8, 160.1, 164.1.

### STEP 3: 5-(3-methoxy-phenyl)-2-methyl-furan-3-carboxylic acid

The compound was obtained following the experimental conditions described in example 5-step 3.

Yield: 98%. The compound was used in the next step without any purification.

¹H NMR (CDCl₃) δ: 2.69 (s, 3H), 3.86 (s, 3H), 6.84 (m, 1H), 6.92 (s, 1H), 7.18-7.33 (m, 3H), 10.83 (brs, 1H).

### STEP 4: 5-(3-methoxy-phenyl)-2-methyl-furan-3-carboxylic acid amide

The compound was obtained following the experimental conditions described in example 1-step 3.

Yield: 74%.

¹H NMR (CDCl₃) δ: 2.67 (s, 3H), 3.85 (s, 3H), 5.68 (brs, 2H), 6.67 (s, 1H), 6.83 (m, 1H), 7.16 (m, 1H), 7.21 (m, 1H), 7.26-7.32 (m, 1H).

ESI-MS m/z [M+Na]⁺ 254 (100), [M+K]⁺ 270, [2 M+Na]⁺ 485.

### STEP 5: 5-(3-hydroxy-phenyl)-2-methyl-furan-3-carboxylic acid amide

The compound was obtained following the experimental conditions described in example 5-step 5.

Yield: 74%.

¹H NMR (CD₃OD) δ: 2.58 (s, 3H), 6.70 (m, 1H), 6.92 (s, 1H), 7.09 (m, 2H), 7.17 (m, 1H); ESI-MS m/z [M+Na]+ 240 (100).

### STEP 6: cyclohexyl-carbamic acid 3-(4-carbamoyl-5-methyl-furan-2-yl)-phenyl ester ST4104

Cyclohexylisocyanate (106 μl, 0.83 mmol) and NEt₃ (116 μl, 0.83 mmol) were added to a solution of 5-(3-methoxy-phenyl)-2-methyl-furan-3-carboxylic acid amide (45.0 mg, 0.21 mmol) in 2.0 ml of dry THF. The reaction mixture was stirred at RT for 16 h. The solvent was removed and purification by means of column chromatography (9:1 CHCl₃/MeOH) afforded 25 mg of the pure product as a colorless solid (34% yield).

¹H NMR (CD₃OD) δ: 1.19-1.39 (m, 5H), 1.65 (m, 1H), 1.79 (m, 2H), 1.96 (m, 2H), 2.61 (s, 3H), 3.38 (m, 1H), 7.02 (m, 2H), 7.39 (m, 2H), 7.50 (m, 1H).

¹³C NMR (CD₃OD) δ: 12.5, 24.9, 25.4, 32.8, 50.5, 104.9, 116.7, 117.3, 120.1, 120.8, 129.6, 131.5, 150.9, 152.0, 155.0, 157.2, 167.3.

ESI-MS m/z [M+Na]⁺ 365, [2 M+Na]⁺ 707.

### Example 9

### (6-phenyl-hexyl)-carbamic acid 3-(4-carbamoyl-5-methyl-furan-2-yl)-phenyl ester ST4105

The compound was obtained following the experimental conditions described in example 8-step 5 using phenylhexylisocyanate (Mor M., et al., J. Med. Chem., 2008, 51, 12, 3487) instead of cyclohexylisocyanate.

Yield: 40%.

¹H NMR (CD₃OD) δ: 1.37 (m, 4H), 1.52-1.63 (m, 4H), 2.58 (m, 5H), 3.16 (t, 2H, J=6.9 Hz), 6.99 (m, 2H), 7.12 (m, 3H), 7.22 (m, 2H), 7.35 (m, 2H), 7.46 (m, 1H).

¹³C NMR (CD₃OD) δ: 12.6, 26.5, 28.8, 29.5, 31.5, 35.7, 40.9, 104.9, 116.7, 117.3, 120.1, 120.8, 125.5, 128.1, 128.2, 129.7, 131.6, 142.7, 150.9, 152.0, 155.8, 157.2, 167.3.

ESI-MS m/z [M+Na]⁺ 443, [M+K]⁺ 459, [2 M+Na]⁺ 863.

### Example 10

### 2-(3-butylcarbamoyloxy-phenyl)-5-methyl-furan-3-carboxylic acid ethyl ester ST4110

### STEP 1: 2-(3-hydroxy-phenyl)-5-methyl-furan-3-carboxylic acid ethyl ester

The compound was obtained following the experimental conditions described in example 5-step 5.

Yield: 60%.

¹H NMR (CD₃OD) δ: 1.30 (t, 3H, J=7.5 Hz), 2.33 (s, 3H), 4.24 (q, 2H, J=7.2 Hz), 6.43 (s, 1H), 6.80 (dd, 1H, J=1.8, 6.9 Hz), 7.21 (t, 1H, J=8.1 Hz), 7.36 (t, 2H, J=7.2 Hz).

### STEP 2: 2-(3-butylcarbamoyloxy-phenyl)-5-methyl-furan-3-carboxylic acid ethyl ester

The compound was obtained following the experimental conditions described in example 5-step 6.

Yield: 81%.

¹H NMR (CDCl₃) δ: 0.94 (t, 3H, J=6.9 Hz), 1.29-1.40 (m, 5H), 1.49-1.59 (m, 2H), 2.32 (s, 3H), 3.25 (q, 2H, J=6.6 Hz), 4.28 (q, 2H, J=6.6 Hz), 5.12 (brs, 1H), 6.42 (s, 1H), 7.14 (d, 1H, J=7.8 Hz), 7.37 (t, 1H, J=8.4 Hz), 7.77 (s, 1H), 7.84 (d, 1H, J=7.5 Hz).

¹³C NMR (CDCl₃) δ: 13.5, 13.9, 14.4, 20.1, 32.1, 41.2, 60.7, 109.2, 115.2, 121.4, 122.4, 125.1, 129.0, 131.4, 151.1, 151.5, 154.7, 154.9, 163.9.

ESI-MS m/z [M+Na]⁺ 368, [2M+Na]⁺ 713.

### Example 11

### 1-(3-butylcarbamoyloxy-phenyl)-1H-pyrrole-3-carboxylic acid undec-10-ynyl ester ST4112

### STEP 1:1-(3-hydroxy-phenyl)-1H-pyrrole-3-carboxylic acid undec-10-ynyl ester

Triphenylphosphine (129 mg, 0.49 mmol), and DIAD (90 ml, 0.49 mmol) were added dropwise to a stirred solution of 1-(3-hydroxy-phenyl)-1H-pyrrole-3-carboxylic acid (100.0 mg, 0.49 mmol) and 10-undecyn-1-ol (90 mL, 0.49 mmol) in 2.0 ml of dry THF at 0° C. The reaction mixture was stirred at RT for 48 h. The solvent was removed under reduced pressure. Saturated aqueous sodium bicarbonate was added to the residue, and the resulting mixture was extracted with AcOEt (3×25 ml), dried over sodium sulfate, filtered and concentrated. Purification was performed by means of flash chromatography (1:9 AcOEt/n-hexane) to afford 120 mg of the desired product (69% yield) as a colorless solid.

¹H NMR (CDCl₃) δ: 1.32-1.55 (m, 12H), 1.70-1.75 (m, 2H), 1.93 (t, 1H, J=2.7 Hz), 2.17 (dd, 2H, J=2.7, 7.2 Hz), 4.25 (t, 2H, J=6.9 Hz), 5.52 (brs, 1H), 6.74 (q, 1H, J=1.8 Hz), 6.79 (dd, 1H J=2.1, 8.1 Hz), 6.92 (t, 1H, J=2.1 Hz), 6.95-7.00 (m, 2H), 7.30 (t, 1H, J=8.1 Hz), 7.66 (dd, 1H, J=1.5, 2.1 Hz).

### STEP 2: 1-(3-butylcarbamoyloxy-phenyl)-1H-pyrrole-3-carboxylic acid undec-10-ynyl ester

The title compound was obtained following the experimental conditions described in example 5-step 6 starting from 1-(3-hydroxy-phenyl)-1H-pyrrole-3-carboxylic acid undec-10-ynyl ester.

Yield: 60%.

¹H NMR (CDCl₃) δ: 0.94 (t, 3H, J=7.2 Hz), 1.30-1.74 (m, 18H), 1.93 (s, 1H), 2.16 (dd, 2H, J=4.5, 6.3 Hz), 3.26 (q, 2H, J=6.3 Hz), 4.22 (t, 2H, J=6.3 Hz), 5.22 (brs, 1H), 6.73 (s, 1H), 6.99 (s, 1H), 7.07 (d, 1H, J=8.1 Hz), 7.19-7.26 (m, 2H), 7.39 (t, 1H, J=8.1 Hz), 7.65 (s, 1H).

¹³C NMR (CDCl₃) δ: 14.0, 18.6, 20.1, 26.2, 28.7, 28.9, 29.1, 29.2, 29.5, 29.6, 32.1, 41.3, 64.3, 68.3, 85.0, 111.9, 114.8, 117.7, 118.6, 120.2, 120.7, 124.5, 130.5, 140.8, 152.2, 154.3, 165.0.

ESI-MS m/z [M+H]⁺ 453, [M+Na]⁺ 475, [M+K]⁺ 491.

### Example 12

### 1-(3-butylcarbamoyloxy-phenyl)-1H-pyrrole-3-carboxylic acid 6-phenyl-hexyl ester ST4114

### STEP 1:1-(3-hydroxy-phenyl)-1H-pyrrole-3-carboxylic acid 6-phenyl-hexyl ester

The title compound was obtained following the experimental conditions described in example 11-step 1 using 6-phenyl-1-hexanol instead of 10-undecyn-1-ol.

Yield: 63%.

¹H NMR (CDCl₃) δ: 1.38-1.48 (m, 4H), 1.49-1.76 (m, 4H), 2.62 (t, 2H, J=6.9 Hz), 4.25 (t, 2H, J=6.9 Hz), 6.73-6.99 (m, 6H), 7.16-7.30 (m, 6H), 7.67-7.68 (m, 1H).

### STEP 2: 1-(3-butylcarbamoyloxy-phenyl)-1H-pyrrole-3-carboxylic acid 6-phenyl-hexyl ester

The title compound was obtained following the experimental conditions described in example 11-step 2 starting from 1-(3-hydroxy-phenyl)-1H-pyrrole-3-carboxylic acid 6-phenyl-hexyl ester.

Yield: 95%.

¹H NMR (CDCl₃) δ: 0.94 (t, 3H, J=7.2 Hz), 1.34-1.81 (m, 11H), 2.62 (t, 3H, J=7.2 Hz), 3.27 (q, 2H, J=6.6 Hz), 4.24 (t, 2H, J=6.6 Hz), 5.21 (t, 1H, J=5.4 Hz), 6.75 (s, 1H), 6.99-7.43 (m, 10H), 7.67 (s, 1H)

¹³C NMR (CDCl₃) δ: 14.0, 20.1, 26.2, 29.0, 29.2, 31.6, 32.1, 36.1, 41.3, 64.3, 112.0, 114.9, 117.7, 118.6, 120.2, 120.8, 124.5, 125.8, 128.5 (2), 128.6 (2), 130.6, 140.8, 142.9, 152.2, 154.3, 165.0.

ESI-MS m/z [M+H]⁺ 461, [M+Na]⁺ 485, [M+K]⁺⁵⁰¹.

### Example 13

### cyclohexyl-carbamic acid 3-(3-carbamoyl-5-methyl-furan-2-yl)-phenyl ester ST4049

Example 6 was synthesized following the experimental conditions described in example 5—step 6, using cyclohexylisocyanate instead of n-butylisocyanate.

Yield: 79%.

¹H NMR (DMSO-d₆) δ: 1.13-1.28 (m, 5H), 1.52-1.59 (m, 1H), 1.67-1.70 (m, 2H), 1.79-1.83 (m, 2H), 2.31 (s, 3H), 3.28-3.30 (m, 1H), 6.48 (s, 1H), 7.04 (dd, 1H, J₁=0.9 Hz, J₂=7.5 Hz), 7.27 (bs, 1H), 7.37 (t, 1H, J=8.1 Hz), 7.63-7.74 (m, 4H).

¹³C NMR (DMSO-d₆) δ: 13.79, 25.24, 25.81, 33.22, 50.44, 109.28, 119.29, 120.57, 122.26, 123.75, 129.73, 131.82, 150.74, 151.46, 151.59, 154.05, 165.69.

### Example 14

### cyclohexyl-carbamic acid 3-(3-carbamoyl-5-methyl-thiophen-2-yl)-phenylester ST4050

The compound was obtained following the experimental conditions described in example 5-step 6 reacting 2-(3-hydroxy-phenyl)-5-methyl-thiophene-3-carboxylic acid amide with cyclohexylisocyanate.

Yield: 72%.

¹H NMR (CD₃OD) δ: 1.22-1.39 (m, 5H), 1.64 (bd, 1H, J=12.6 Hz), 1.78 (bd, 2H, J=12.0 Hz), 1.94 (bd, 2H, J=10.5 Hz), 2.47 (s, 3H), 3.38-3.48 (m, 1H), 6.97 (m, 1H), 7.09 (d, 1H, J=8.1 Hz), 7.21 (s, 1H), 7.30-7.41 (m, 2H).

¹³C NMR (CD₃OD) δ: 13.71, 24.98, 25.42, 32.80, 50.48, 121.46, 122.06, 125.57, 126.79, 129.30, 132.89, 134.67, 139.75, 141.39, 151.5, 155.03, 167.13.

ESI-MS m/z [M+Na]⁺ 381, [2M+Na]⁺ 740.

### Preparation 1: 11-isocyanato-undec-1-yne

Undec-10-ynylamine (Crisp G. T., et al., Tetrahedron, 1997, 53, 4, 1505, 514 mg) was dissolved in 33 ml of CH₂Cl₂. The solution was cooled to 0° C. and a saturated aqueous solution of sodium bicarbonate (30 ml) was added. The biphasic mixture was stirred for 10 min at 0° C., the layers were allowed to separate, and a solution of phosgene (20% in toluene, 3.23 ml) was added directly to the organic layer via syringe. After 15 min, the aqueous layer was extracted with CH₂Cl₂. The combined organic layers were dried over sodium sulfate, filtered and concentrated. The compound was used without any further purification.

Yield: 94%.

¹H NMR (CDCl₃) δ: 1.27 (m, 4H), 1.51 (m, 4H), 2.51 (t, 2H, J=7.7 Hz), 3.14 (t, 2H, J=6.6 Hz), 7.07 (m, 3H), 7.17 (m, 2H).

### Biological Results

FAAH assay

The compounds of the present invention show affinity and inhibit the enzymatic activity of the fatty acid amide hydrolase enzyme.

The assay of FAAH (EC 3.5.1.4) was performed by measuring the release of [1-¹⁴C]AA from [1-¹⁴C]AnNH (52 mCi/mmol), using RP-HPLC. Also [³H]AnNH (205 Ci/mmol) could be used as substrate, measuring the release of [³H]AA under the same experimental conditions described below for [1-¹⁴C]AnNH. Compounds of the invention, at various concentrations, were added in 200 μl hydrolase assay buffer (50 mM Tris-HCl, pH 9.0), in 2-ml Eppendorf tubes, 20 min before adding [1-¹⁴C]AnNH₂, up to a concentration of 10 μM. The reaction was initiated by the addition of mouse brain homogenate (40 μg), and after incubation at 37° C. for 15 min it was stopped by the addition of 800 μl ice-cold methanol/chloroform (2:1, v/v) with vortexing. This mixture was allowed to stand at room temperature for 30 min, then 240 μl chloroform and 240 μl water were added with vortexing. After 10 min at room temperature, the mixture was centrifuged at 3000 g for 5 min, the upper aqueous layer was removed by suction and the lower organic phase was dried by spinning the samples in a DNA MINI speedvac (Heto-Holten, Denmark), at 100 mbar and 30° C. for 30 min. The residue was dissolved into 50 μl methanol and subjected to RP-HPLC analysis for AA quantitation, as detailed below. FAAH specific activity was expressed as pmol AA released/min/mg protein. Kinetic studies were performed by Lineweaver-Burk analysis, using [1-¹⁴C]AnNH, [1-¹⁴C]ODNHEtOH, or [1-¹⁴C]ODNH₂ in the concentration range 0-12 μM. Fitting of the experimental points by a linear regression programme (Kaleidagraph 3.0) yielded straight lines with r values >0.97.

TABLE 1

| Examples | ST Number | IC₅₀ (nM) | Ki (nM) |
|---|---|---|---|
| 1 | 3910 | ++ | +++ |
| 2 | 3911 | ++++ | ++++ |
| 3 | 3912 | ++ | +++ |
| 4 | 3913 | ++++ | ++++ |
| 5 | 4067 | + | ++ |
| 6 | 4068 | ++++ | ++++ |
| 7 | 4069 | ++++ | ++++ |
| 8 | 4104 | ++ | ND |
| 9 | 4105 | +++ | ++++ |
| 10 | 4110 | ++ | ND |
| 11 | 4112 | ++ | ND |
| 12 | 4114 | ++ | ND |
| 13 | 4049 | ++ | ND |
| 14 | 4050 | ++ | ND |

[++++] [IC₅₀] < 10 nM and/or [Ki] < 10 nM
[+++] 10 nM < [IC₅₀] < 100 nM and/or 10 nM < [Ki] < 100 nM
[++] 100 < [IC₅₀] < 500 nM and/or 100 < [Ki] < 500 nM
[+] 500 < [IC₅₀] < 5000 nM and/or 500 < [Ki] < 5000 nM
ND: not determined

### Selectivity Profile

The compounds of the present invention were also evaluated with regard to their selectivity profile against the following targets: AMT, NAPE-PLD, MAGL, DAGL, CB1/CB2 and TRPV1 according to the procedures described in Maccarrone M., et al., J. Biol. Chem., 2000, 275, 13484, Fezza F., et al., Anal Biochem., 2005, 339, 113, Dinh T. P., et al., 2002, Proc. Natl. Acad. Sci. 99, 10819, Bisogno T., et al., 2003, J. Cell Biol., 163, 463, Maccarrone M., et al., J. Biol. Chem., 2000, 275, 31938, Ross R. A., et al., Br. J. Pharmacol., 2001, 132, 631. The results are shown in table 2.

TABLE 2

| | ST3913^{a} | ST4069 | ST4105 |
|---|---|---|---|
| CB1R | −−−− | −−−− | −−−− |
| CB2R | −−−− | −−−− | −−−− |
| TRPV1 | −−−− | −−−− | −−−− |
| NAPE-PLD | −−−− | −−−− | −−−− |
| AMT | 80% @ 1 μM | 60% @ 1 μM | 80% @ 1 μM |
| DAGL | −−−− | −−−− | −−−− |
| MAGL | −−−− | −−−− | −−−− |

[−−−−] 1000 times [IC₅₀] with an inhibitory activity on the target <60%
[−−−] 100 times [IC₅₀] with an inhibitory activity on the target <60%
[−−] 10 times [IC₅₀] with an inhibitory activity on the target <60%
[−] 5 times [IC₅₀] with an inhibitory activity on the target <60%

### Analgesia In Vivo Activity

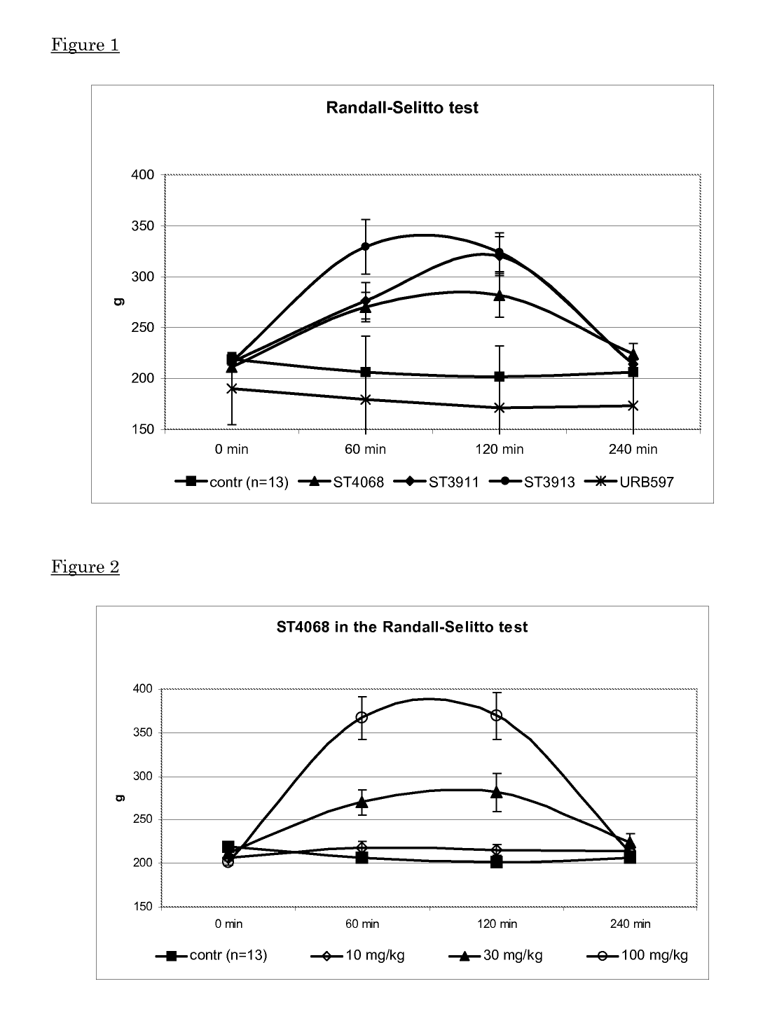

The paw withdrawal test was used to assess mechanical hyperalgesia. The nociceptive threshold, expressed in grams, was measured by applying increasing pressure to the left and right hind paws using a Randall-Selitto analgesimeter (Ugo Basile, Varese, Italy). The parameter used to quantify the nociceptive threshold was defined as the pressure (grams) at which the rat withdrew its paw. Rats were habituated to the testing procedures and handling by the investigator in the week prior to the experiment. Acute oral treatment with ST4068, ST3911 and ST3913 at a dose of 30 mg/kg demonstrated a significant analgesic activity, meanwhile the well-known reference compound URB597 at a dose of 50 mg/kg did not show any activity (FIG. 1).

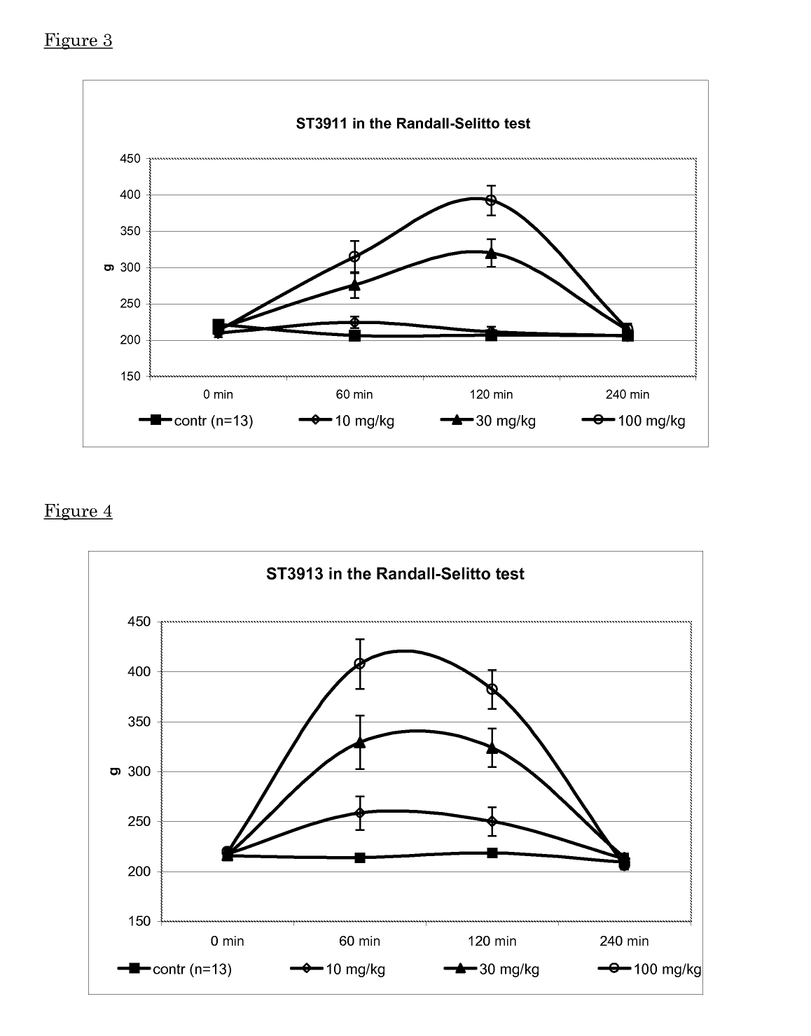

Dose response experiments at 10, 30 and 100 mg/kg were also conducted with ST4068, ST3911 and ST3913. All three compounds did exert analgesic activity at 30 and 100 mg/kg dose regimen, the latter dosage conferring a higher effect (FIGS. 2, 3 and 4).

1. A compound having the general formula I 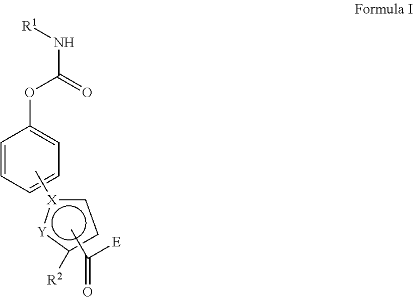
wherein,
R¹ is H, (C₁-C₄)-alkyl, (C₃-C₆)-cycloalkyl, (C₁-C₆)-alkyl substituted with aryl or (C₂-C₅)-alkynyl;
X is C or N;
Y is CH, O or S;
R² is H or (C₁-C₄)-alkyl;
E is NR³R⁴ or OR⁵;
R³ and R⁴, the same or different are H or (C₂-C₆)-alkyl optionally substituted with aryl;
R⁵ is (C₂-C₆)-alkyl optionally substituted with aryl or with (C₂-C₅)-alkynyl;
wherein the cycle containing the radicals X and Y is a heteroaromatic ring;
its optically active forms such as enantiomers, diastereomers and its racemate forms, as well as pharmaceutically acceptable salts thereof;
with the proviso that when X is N, Y is CH.

2. The compound according to claim 1, wherein R¹ is (C₁-C6)-alkyl substituted with aryl or (C₂-C₅)-alkynyl.

3. The compound according to claim 1, wherein R³ and R⁴ are H.

4. The compound according to claim 1, selected from the group consisting of: butyl-carbamic acid 3-(3-carbamoyl-pyrrol-1-yl)-phenyl ester, undec-10-ynyl-carbamic acid 3-(3-carbamoyl-pyrrol-1-yl)-phenyl ester, cyclohexyl-carbamic acid 3-(3-carbamoyl-pyrrol-1-yl)-phenyl ester, (6-phenyl-hexyl)-carbamic acid 3-(3-carbamoyl-pyrrol-1-yl)-phenyl ester, butyl-carbamic acid 3-(3-carbamoyl-5-methyl-furan-2-yl)-phenyl ester, (6-phenyl-hexyl)-carbamic acid 3-(3-carbamoyl-5-methyl-furan-2-yl)-phenyl ester, (6-phenyl-hexyl)-carbamic acid 3-(3-carbamoyl-5-methyl-thiophen-2-yl)-phenyl ester, cyclohexyl-carbamic acid 3-(4-carbamoyl-5-methyl-furan-2-yl)-phenyl ester, (6-phenyl-hexyl)-carbamic acid 3-(4-carbamoyl-5-methyl-furan-2-yl)-phenyl ester, 2-(3-butylcarbamoyloxy-phenyl)-5-methyl-furan-3-carboxylic acid ethyl ester, 1-(3-butylcarbamoyloxy-phenyl)-1H-pyrrole-3-carboxylic acid undec-10-ynyl ester, 1-(3-butylcarbamoyloxy-phenyl)-1H-pyrrole-3-carboxylic acid 6-phenyl-hexyl ester, cyclohexyl-carbamic acid 3-(3-carbamoyl-5-methyl-furan-2-yl)-phenyl ester and cyclohexyl-carbamic acid 3-(3-carbamoyl-5-methyl-thiophen-2-yl)-phenylester.

5-6. (canceled)

7. Method of treating a pathological state comprising
administering to a patient in need thereof an effective amount of compounds according to claim 1, for the treatment of a pathological state chosen from the group consisting of a neurological disorder, disease of energy metabolism, cardiovascular and respiratory disorders,
gastrointestinal and liver disorders, retinopathy, cancer and musculoskeletal disorders, and
treating said patient of said pathological state.

8. Method according to claim 7 where the disorder is a neurological disorder.

9. Method according to claim 8 where the disorder is anxiety.

10. Method according to claim 8 where the disorder is neuropathic pain.

11. Method according to claim 8 where the disorder is Parkinson's disease.

12. A pharmaceutical composition containing at least one compound according to claim 1 as the active ingredient in mixtures with at least one pharmaceutically acceptable vehicle and/or excipient.

13. A method for inhibiting FAAH comprising the step of administering to a mammal afflicted with a pathological state for which the modulation of FAAH activity would result at improving the health of the patient, an effective amount of a compound of claim 1.

14. A process for synthesizing compounds of claim 1 comprising reacting compound of formula II 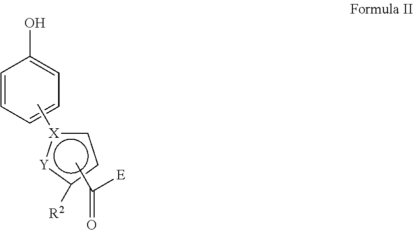
wherein
X is C or N;
Y is CH, O or S;
R² is H or (C₁-C₄-alkyl
E is NR³R⁴ or OR⁵;
R³ and R⁴, the same or different are H or (C₂-C₆)-alkyl optionally substituted with aryl;
R⁵ is (C₂-C₆)-alkyl optionally substituted with aryl or with (C₂-C₅)-alkynyl;
wherein the cycle containing the radicals X and Y is a heteroaromatic ring;
its optically active forms such as enantiomers, diastereomers and its racemate forms, as well as pharmaceutically acceptable salts thereof;
with the proviso that when X is N, Y is CH,
with compounds of formula III
R¹—N═C═O Formula III
wherein R¹ is H, (C₁-C₄)-alkyl, (C₃-C₆)-cycloalkyl, (C₁-C₆)-alkyl substituted with aryl or (C₂-C₅),
in a polar solvent in the presence of a base such as NEt₃.

15. A process for preparing a pharmaceutical composition comprising bringing a compound according to claim 1 and a pharmaceutically acceptable vehicle and/or excipient into intimate admixture.

16. Method according to claim 7, wherein said effective amount comprises from 0.01 mg/kg to 100 mg/kg.

17. Method according to claim 16, wherein said effective amount comprises from 0.05 mg/kg to 50 mg/kg.
